(19) United States
(12) Patent Application Publication
(10) Pub. No.: US 2026/0027317 A1
(43) Pub. Date: Jan. 29, 2026

(54) MASK WITH A VALVE

(71) Applicant: DCSTAR INC, New York, NY (US)

(72) Inventor: David LUO, New York, NY (US)

(21) Appl. No.: 18/786,021

(22) Filed: Jul. 26, 2024

(51) Int. Cl. A61M 16/06; A61M 16/20

(52) U.S. Cl. CPC A61M 16/0616; A61M 16/208; A61M 2205/0216; A61M 2205/0233; A61M 2205/025; A61M 2210/0618; A61M 2210/0625

(57) ABSTRACT

A mask with a valve which provides protection for a device with the mask in multiple fields. The mask includes three parts: a mask body, a connecting part, and a valve assembly. The mask body has a first side and a second side, configured to at least partially cover the entrance of a nasal and/or oral airway of the user; the connecting part has a third side and a fourth side, configured to connect to the mask body and allow gas to enter or exit the mask body; the valve assembly is provided on a through passage which is formed by extending on both sides of the connecting part and is configured to allow airflow to pass through the connecting part in one direction only. Moreover, the disclosure achieves more effective use of the mask by defining the valve assembly.

### TECHNICAL FIELD

This disclosure pertains to a mask with a valve, configured to enhance the reliability of a device with the mask in multiple fields and to ensure the effectiveness of its use.

### BACKGROUND

Masks are essential in several areas of daily life. In the medical field, masks are commonly used for mechanical ventilation therapy of users or for removing obstructions from a user' s airway during emergency resuscitation to provide urgent respiratory support. They are also used in surgical procedures to administer anesthesia gases. In home care, masks can be used with associated devices to treat or improve symptoms related to respiratory disorders such as Chronic Obstructive Pulmonary Disease (COPD), Obstructive Sleep Apnea (OSA), and asthma. Additionally, masks find applications in emergency response, athletic training, and scientific research, which proves their importance and versatility across various industries and their necessity in multiple fields.

Common types of masks include nasal pillows, nasal masks, oral masks, and full-face masks. Nasal pillows are the lightest and most compact, having two protrusions that fit into the nostrils with minimal contact with the face. Nasal masks are larger than nasal pillows, configured to deliver airflow into the nostrils without entering them and only covering the nose. Face masks supply airflow to both the nostrils and mouth, covering both the nose and mouth of the user. Full-face masks provide the most comprehensive and sealed airflow supply, covering from the bridge of the nose to the chin, encompassing a larger part of the face. Different mask types correspond to various use cases and demographics, and the choice of mask also depends on comfort needs, pressure needs, application area, and facial features. Some masks are set to interface with one-way valves, allowing air to flow only in one direction and preventing flow in the opposite direction. These masks are often used in anti-choking emergencies (where one-way valves prevent pushing blockages deeper into the airways during suction), anesthesia masks (which provide anesthetic gases during surgeries while ensuring unidirectional flow to prevent backflow and leakage into the operating room), and in chemical and pharmaceutical processes (where one-way valves prevent the inhalation of toxic substances).

Common valve types used with masks include diaphragm valves, ball valves, spring valves, and active valves. However, in existing devices available on the market, the mask and valve are separate components, often resulting in poor performance. During the compression of expelled air, it is possible for the air to be pushed into the user's airway, further exacerbating the severity of the user's condition.

### SUMMARY

This disclosure addresses the deficiencies above and provides a mask for use in multiple fields that enhances effectiveness and prevents misuse.

In one embodiment, a mask with a valve is provided. The mask with a valve includes a mask body, a connecting part, and a valve assembly. The mask is configured to provide protection for a device and includes a mask body. The mask body has a first side configured to be in contact with a face of a user and a second side opposite the first side. The mask body is configured to at least partially cover an entrance of a nasal and/or oral airway of the user. The mask includes a connecting part that has a third side adjacent to the second side and a fourth side opposite the third side. The connecting part is configured to expel airflow from the mask body or deliver the airflow into the mask body. The mask also includes a valve assembly including the valve, and the valve assembly is provided on a through passage formed by extending on both sides of the connecting part. The valve assembly is configured to allow the airflow to pass through the connecting part in one direction only.

In one embodiment, at least one cross-section of the connecting part has a shape of a circle, an ellipse, or a polygon.

In one embodiment, at least part of the connecting part has an unbent shape.

In one embodiment, the valve assembly has two ends, and when there is no airflow, a projected area of the two ends on a horizontal plane differs.

In one embodiment, the valve assembly includes one or more of the following materials: silicone, plastic, or metal.

In one embodiment, the first side of the mask body includes a flexible pad. The flexible pad is configured to form a seal with the face of the user.

In another embodiment, a mask with a valve is provided. The mask with a valve includes a mask body, a connecting part, and a valve assembly. The mask is configured to provide protection for a device and includes a mask body. The mask body has a first side configured to be in contact with a face of a user and a second side opposite the first side. The mask body is configured to at least partially cover an entrance of a nasal and/or oral airway of the user. The mask also includes a connecting part that has a third side adjacent to the second side and a fourth side opposite the third side. The connecting part is configured to expel airflow from the mask body or deliver the airflow into the mask body. The mask also includes a valve assembly including the valve, and the valve assembly is provided on a through passage formed by extending on both sides of the connecting part. The valve assembly is configured to allow the airflow to pass through the connecting part in one direction only and an opening is provided on the second side of the mask body. The projected area of the valve assembly on a horizontal plane is greater than 30% of the area of the opening.

In one embodiment, the outer perimeter of the valve assembly is greater than the perimeter of the opening.

In one embodiment, the outer perimeter of the valve assembly is less than or equal to the perimeter of the opening.

In one embodiment, the valve assembly is configured to allow the airflow to flow from the first side towards the fourth side only.

In one embodiment, at least part of the valve assembly is connectable and secured to the connecting part. The connection of at least part of the valve assembly to the connecting part is achieved through one or more of the following: adhesive bonding, welding, or mechanical structural connection.

In yet another embodiment, a mask with a valve is provided. The mask with a valve includes a mask body, a connecting part, and a valve assembly. The mask is configured to provide protection for a device with the mask in multiple fields and includes a mask body. The mask body has a first side configured to be in contact with a face of a user and a second side opposite the first side. The mask body is configured to at least partially cover an entrance of a nasal and/or oral airway of the user. Additionally, the mask includes a connecting part with a third side adjacent to the second side and a fourth side opposite the third side. The connecting part is configured to expel airflow from the mask body or deliver the airflow into the mask body. The mask also includes a valve assembly including the valve, and the valve assembly is provided on a through passage formed by extending on both sides of the connecting part. The valve assembly is configured to allow the airflow to pass through the connecting part in one direction only. During a process of unidirectional airflow, the valve assembly forms at least one airflow port, and a maximum area of the at least one airflow port is less than or equal to 38.485 cm².

In one embodiment, a central axis of the valve assembly coincides with or is parallel to a central axis of the connecting part.

In one embodiment, the distance from the valve assembly to the first side of the mask body is greater than one-tenth of the distance from the second side of the mask body to the first side.

In one embodiment, the distance between the valve assembly and the fourth side of the connecting part is less than or equal to the distance between the first side of the mask body and the fourth side of the connecting part.

In one embodiment, the mask body is configured to have a universal size.

In a further embodiment, a mask with a valve is provided. The mask includes a mask body, a connecting part, and a valve assembly, configured to provide protection for a device with the mask in multiple fields. The mask body has a first side configured to be in contact with a face of a user and a second side opposite the first side. The mask body is configured to at least partially cover an entrance of a nasal and/or oral airway of the user. The connecting part, having a third side adjacent to the second side and a fourth side opposite the third side, is configured to expel airflow from the mask body or deliver the airflow into the mask body. Additionally, the mask includes a valve assembly including the valve, and the valve assembly is provided on a through passage formed by extending on both sides of the connecting part. The valve assembly is configured to allow the airflow to pass through the connecting part in one direction only and includes at least one thin region. The at least one thin region has a thickness thinner than other parts of the valve assembly and is configured to deform to allow unidirectional airflow through the valve assembly. The thickness of at least part of the at least one thin region is less than or equal to 5 mm.

In one embodiment, the valve assembly includes at least one slit, and the at least one slit is provided on a middle of the at least one thin region and/or at a joining area of multiple thin regions.

In one embodiment, the at least one thin region is configured so that it does not damage its material or performance during deformation.

In one embodiment, at least part of the at least one thin region includes a flexible material.

In one embodiment, the valve assembly and the connecting part are not integrally formed.

In one embodiment, the valve assembly and the connecting part are at least partially integrally formed.

The implementation of a mask with a valve provided by this disclosure at least includes the following benefits:
1) The mask of this disclosure is versatile and applicable to multiple fields, including medical emergencies and home care. In the existing market, devices such as anti-choking devices, sputum suction devices, and emergency respirators all involve configurations where valves are used in conjunction with masks. However, there is room for improvement for these existing devices. For example, the existing anti-choking devices typically consist of a negative pressure generator, a one-way valve, and a mask. However, the one-way valve is not set up to connect directly with the mask, resulting in only a single channel at the junction between the negative pressure generator and the mask without any one-way valve to isolate the two. When the anti-choking device is activated, it first needs to expel excess air from the negative pressure generator to create a vacuum. At this time, because the channel between the negative pressure generator and the mask is not isolated, the air expelled by the negative pressure generator can easily enter the mask. This process contradicts the original purpose of the anti-choking device as it forces air into the mask that should be evacuating internal air. This forced air entry can push obstructions deeper into the user's airway and, in extreme cases, cause severe injuries like throat lacerations, presenting a hazard to the user. Therefore, by incorporating a one-way valve at an appropriate location on the mask and refining the design of the one-way valve to enhance its effectiveness and reliability, the mask provided by this disclosure can be used in existing devices to ensure the safety of devices like anti-choking systems. It guarantees the correct airflow during the vacuum creation process and prevents operational errors during this phase. Given the extensive use of one-way valves in conjunction with masks across multiple fields, the one-way valve-equipped mask of this disclosure improves various existing products, increasing the overall reliability of devices in multiple fields and enhancing the product experience for users.
2) In some devices within the aforementioned fields, there exists a one-way valve between the negative pressure generator and the mask. In such cases, using the mask with a one-way valve from this disclosure provides a secondary level of protection for the device. Specifically, the mask of this disclosure not only prevents the backflow of gases during the vacuum formation process but also maintains the stability of the vacuum more efficiently. In the event of an accidental device malfunction, the mask with the valve offers additional safety, making the device more secure and reliable. This dual mechanism is particularly crucial in high-demand or highly specialized fields, providing users with a safer and more effective respiratory support solution.
3) This disclosure also offers advantages when the valve and mask are integrated as a single module. a. For users, simply replacing the mask can enhance the overall device's safety, making it a very cost-effective choice. This is especially true for devices without isolation between the negative pressure generator and the mask, as replacing the original mask with a mask with a valve brings safety for users. b. Furthermore, when the valve and mask form a combinable module with a detachable connection, it allows users to choose between masks with or without a valve, thereby broadening their options and extending the adaptability of the mask of this disclosure to different application scenarios and needs. c. Moreover, compared to the combination of the valve with the negative pressure generator, the combination of the valve with the mask facilitates easier cleaning for the user. Due to the valve typically having more hygiene dead spots compared to other parts of the device, when cleaning is required, the mask itself, with its simple structure and bidirectional openings, makes the valve connected to the mask easier to clean thoroughly. This ensures that the device can maintain a good sanitary condition before and after each use, thereby enhancing the convenience of use.

### BRIEF DESCRIPTION OF THE DRAWINGS

FIG. 1 is a three-dimensional schematic diagram of the mask in accordance with one embodiment;

FIG. 2 is a three-dimensional schematic diagram of the through passage in the connecting part in multiple embodiments;

FIG. 3 is a three-dimensional schematic diagram of the mask when the valve assembly allows gas flow in accordance with one embodiment;

FIG. 4 is a three-dimensional schematic diagram where the valve assembly is detachable within the mask in multiple embodiments;

FIG. 5 is a three-dimensional schematic diagram of the area of the airflow port of the valve assembly in multiple embodiments;

FIG. 6 is a three-dimensional sectional view of a mask with a valve in multiple embodiments;

FIG. 7 is a three-dimensional schematic diagram comparing the projected area of the valve assembly in the horizontal plane and the area of the opening of the second side in multiple embodiments;

FIGS. 8A, 8B, and 8C are three-dimensional schematic diagrams showing different positions of the valve assembly in the through passage in multiple embodiments;

FIG. 9 is a three-dimensional schematic diagram of the valve assembly's flexible material forming a certain height without folds in accordance with one embodiment;

FIGS. 10A and 10B are three-dimensional schematic diagrams showing the perimeter of the valve assembly being larger or smaller than the perimeter of the opening in accordance with one embodiment;

FIG. 11 is a three-dimensional schematic diagram where the flexible part of the valve assembly does not form a height in accordance with one embodiment.

### DETAILED DESCRIPTION

To make the objectives, features, and advantages of this disclosure more clear and comprehensible, the specific embodiments of this disclosure are described in detail with reference to the accompanying drawings. Extensive specific details are provided in the description below to facilitate a comprehensive understanding of the disclosure. However, the disclosure can be implemented in many different ways other than those described here, and those skilled in the art can make similar modifications without departing from the essence of the disclosure; thus, this disclosure is not limited by the specific embodiments disclosed below.

Compared to existing devices on the market that use masks in conjunction with valves, this disclosure provides a mask with a valve that enhances the overall effectiveness of the device and increases safety for the user. The mask with a valve can be used to replace masks in devices where there is no isolation between the negative pressure generator and the mask, as well as in devices where there is a valve between the negative pressure generator and the mask, but the mask does not directly connect to the valve. Devices with the replaced mask provide greater safety assurance for the user. Furthermore, the mask of this disclosure also aids in cleaning and increases the choice and adaptability of masks for users. Simply replacing the mask to enhance the overall device's safety, compared to replacing the entire device, is also a more economical and preferable choice for users.

This disclosure provides a mask with a valve 1, configured to enhance the overall reliability of devices across multiple fields and prevent operational errors.

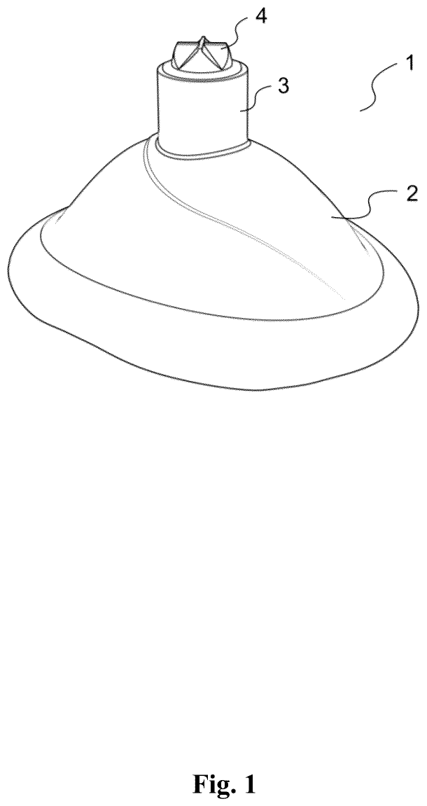

Specifically, please refer to FIGS. 1 through 4, the mask 1 of this disclosure includes three parts: the mask body 2, the connecting part 3, and the valve assembly 4. By defining the form of connection between the connecting part 3 and the valve assembly 4 and the forms and sizes of the valve assembly 4, a more stable and effective mask 1 is achieved. The mask 1 of this disclosure can be used in various devices across different fields, such as anti-choking devices, sputum suction devices, and emergency respirators, all of which utilize a mask in conjunction with other components. Typically, the masks in these devices are detachable from other parts of the devices, which brings convenience for replacing different sizes of the mask body 2. Therefore, the mask 1 of this disclosure can be used in these various devices to enhance their reliability and achieve higher effectiveness. Furthermore, the devices described below can be interpreted as devices that use the mask body 2 in conjunction with the valve assembly 4, i.e., devices that can utilize the mask 1 provided by this disclosure. In most cases, the mask 1 of this disclosure is used in conjunction with a negative pressure generator.

The mask body 2 is the part that directly contacts the user's face, having a first side 21 that is configured to contact the user's face and a second side 22 opposite to the first side 21. The mask body 2 is configured to at least partially cover the user's entrance of a nasal and/or oral airway. Devices with the mask 1 of this disclosure require the mask body 2 to cover the user's face during use. The mask body 2 can be designed in different sizes according to the face sizes of different users and is generally configured in a universal size. The mask body 2 must both withstand the pressure of gases expelled by the negative pressure generator and contain a certain volume of gas, which means that the mask body 2 needs to be made of relatively rigid materials; and the mask body 2 must also contact the user's face to create a certain level of sealing, thus given the user's facial comfort and sealing requirements, the part of the mask body 2 that contacts the user's face needs to be made of more flexible materials. Therefore, the mask body 2 uses more rigid materials at positions away from the user's face (i.e., the second side 22) to form an umbrella shape, and the first side 21 of the mask body 2 includes a flexible pad configured to form a seal with the user's face.

The mask 1 further includes a connecting part 3. The mask 1 detaches from or combines with other parts of the device through the connecting part 3. The connecting part 3 has a third side 31 adjacent to the second side 22 and a fourth side 32 opposite the third side 31. The connecting part 3 is configured to expel airflow from the mask body 2 or to deliver airflow into the mask body 2. The connecting part 3 extends on both sides to form a through passage 331 that houses the valve assembly 4, and the solid parts within the through passage 331 form a cavity 33 that allows gas flow. This cavity 33 is configured to either deliver gas into the mask body 2 or extract gas from the cavity of the mask body 2. To facilitate connection with components on the third side 31 and the fourth side 32 and ensure smooth gas flow, at least a part of the cavity 33 is set as an unbent shape (i.e., allowing gas to flow in a straight line within the cavity). The cavity 33 typically is in the shape of a vertical cylindrical tube; in other cases, the cavity 33 can be configured in any shape other than a cylindrical tube, and the cavity 33 can also have a curved form. Therefore, the cross-section of the cavity 33 is set to be one of a circle, an ellipse, or a polygon. The connection between the connecting part 3 and the mask body 2 can be either detachable or integrally formed. In some cases, the second side 22 of the mask body 2 coincides with the third side 31 of the connecting part 3, which is common when the connecting part 3 and the mask body 2 are integrally formed. In other cases, the second side 22 of the mask body 2 and the third side 31 of the connecting part 3 may not coincide. Regardless of the positional relationship and connection between the connecting part 3 and the mask body 2, there will always be at least one opening 221 between them, configured to connect the cavity 33 and the mask 1.

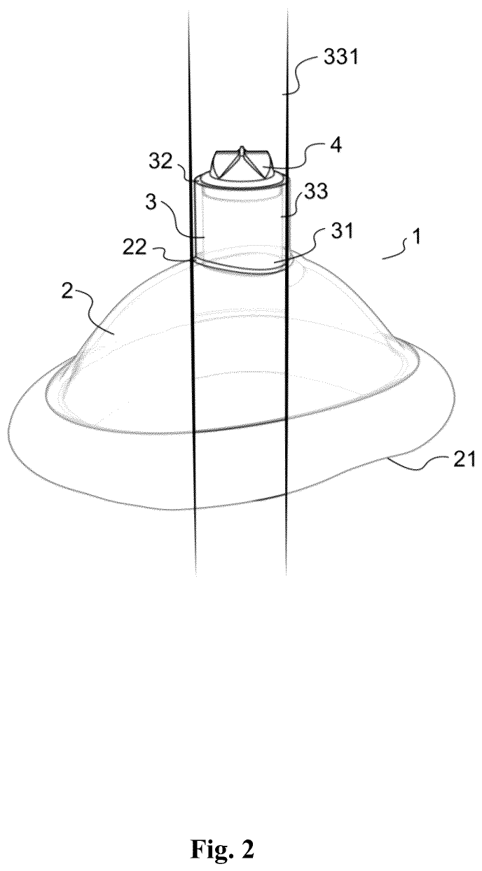

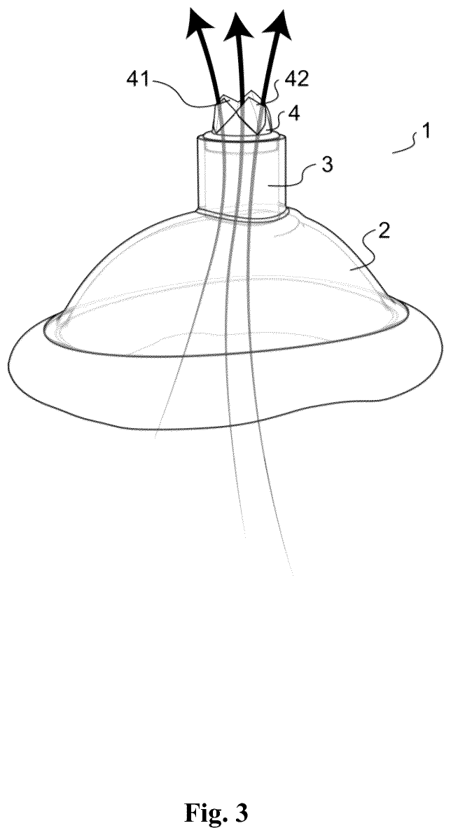

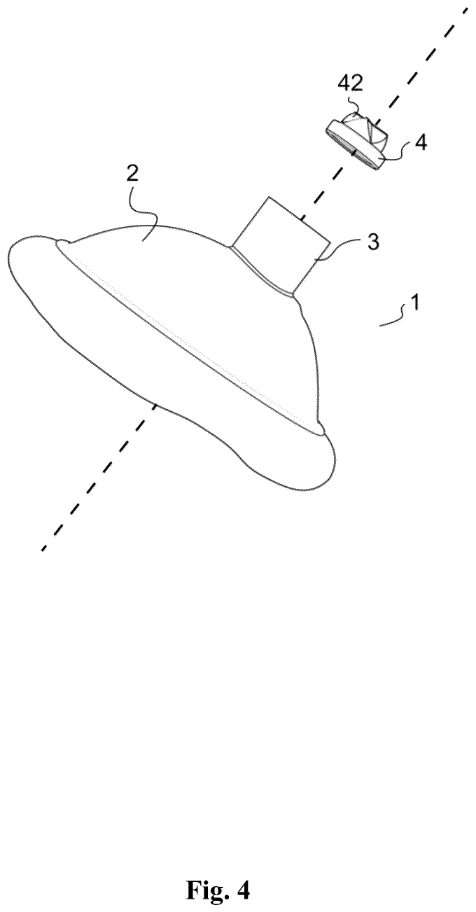

The valve assembly 4 is the core component of the mask 1 in this disclosure, provided on the through passage 331 formed by extending on both sides of the connecting part 3. The valve assembly 4 is configured to allow airflow to pass through the connecting part 3 in only one direction (as shown in FIGS. 2 and 3). The through passage 331 formed by extending on both sides of the connecting part 3 can be understood as the passage that is formed by elongating the connecting part 3 on both sides along the original curvature of the inner wall of the cavity 33 at the third side 31 and the fourth side 32, and the valve assembly 4 is provided on the through passage 331. The valve assembly 4 includes one or more materials selected from silicone, plastic, and metal. The valve assembly 4 can be integrally formed from a single material or can be a combination of multiple materials. When the valve assembly 4 includes multiple materials, it can be a combination of multiple structures. Likewise, even when the valve assembly 4 only includes a single material, it can still be a combination of multiple structures. Furthermore, the valve assembly 4 is configured to be at least partially connected and secured to the connecting part 3, and they are not integrally formed (as shown in FIG. 4). There are two non-integral formation scenarios for the valve assembly 4 and the connecting part 3: either they are detachably connected, or they are configured as two separate but non-detachable parts. Alternatively, the valve assembly 4 and the connecting part 3 can be at least partially integrally formed. When the valve assembly 4 and the connecting part 3 are detachably connected, the connection can be achieved through various methods. Specifically, the connection between at least part of the valve assembly 4 and the connecting part 3 can be realized by adhesive bonding, welding, or mechanical structural connections, alone or in combination. The direction of gas flow allowed by the valve assembly 4 can be configured to permit airflow only into the cavity formed by the mask body 2, meaning the valve assembly 4 is configured to allow airflow only from the fourth side 32 towards the first side 21. Conversely, it can be configured to permit only the expulsion of gas from the cavity formed by the mask body 2, meaning the valve assembly 4 is configured to allow airflow only from the first side 21 towards the fourth side 32.

Detailed embodiments are presented below to elucidate the configurations of the mask 1 provided by this disclosure.

### EMBODIMENT 1

In this embodiment, the mask 1 includes: a mask body 2, a connecting part 3 and a valve assembly 4. The mask body 2 has a first side 21 that is configured to contact the user's face and a second side 22 opposite to the first side 21. The mask body 2 is configured to at least partially cover the user's entrance of a nasal and/or oral airway.

The connecting part 3 has a third side 31 to connect to the second side 22 and a fourth side 32 opposite to the third side 31. The connecting part 3 is configured to expel airflow from the mask body 2 or to deliver airflow into the mask body 2. The valve assembly 4, provided on the through passage 331 formed by extending on both sides of the connecting part 3, is configured to allow airflow to pass through the connecting part 3 in one direction only.

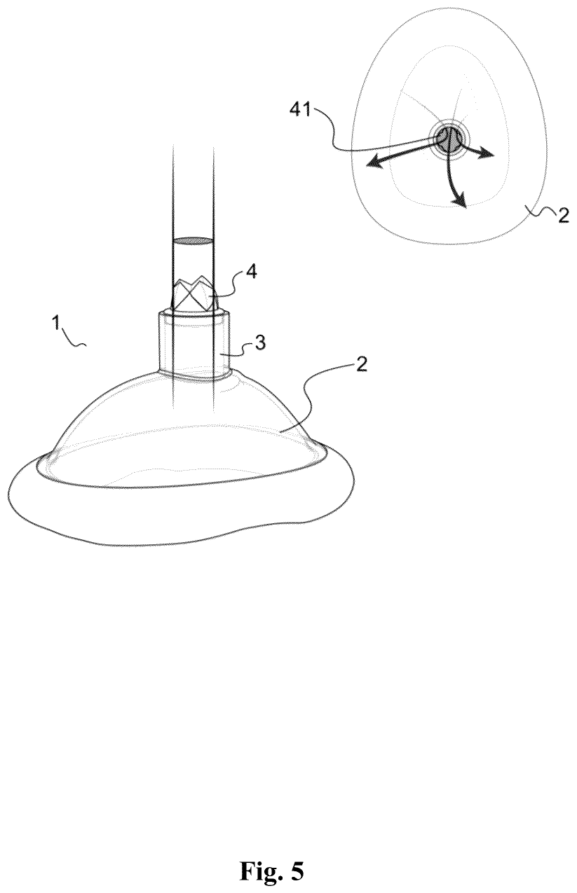

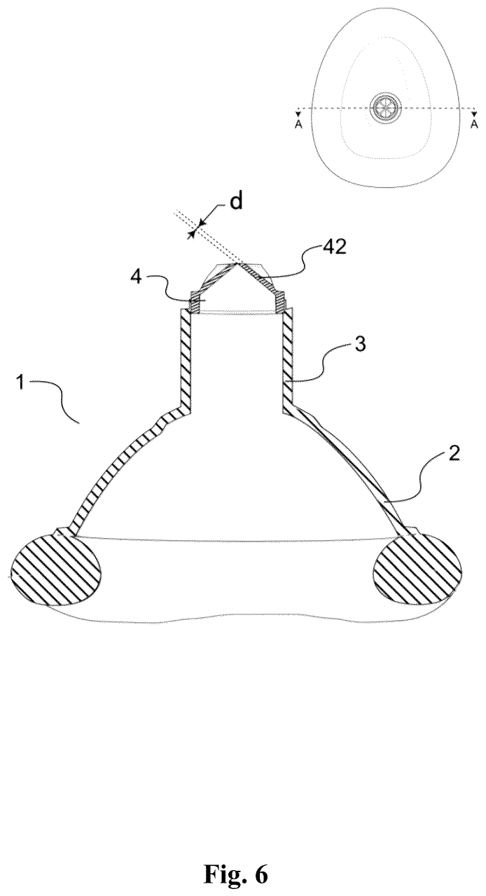

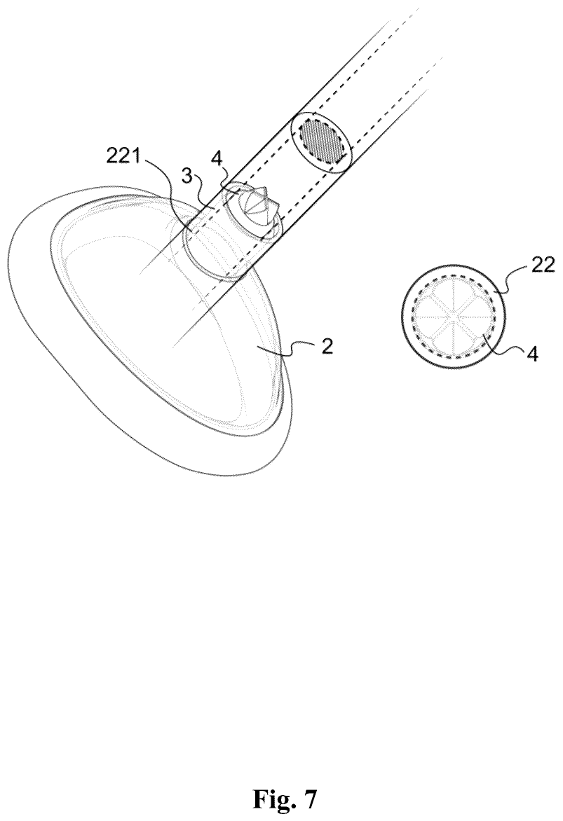

In this embodiment, the valve assembly 4 at least partially includes flexible material, and the valve assembly 4 forms at least one airflow port 41 during the process of allowing unidirectional airflow. The maximum area of the airflow port 41 is less than or equal to 38.485 cm² (as shown in FIG. 5). The formation of the airflow port 41 mainly relies on the deformation of thin regions 42 in the valve assembly 4, meaning the valve assembly 4 has at least one thin region 42. The thin region 42 has a thickness thinner than other parts of the valve assembly 4 and is configured to deform to allow unidirectional airflow through the valve assembly 4. The valve assembly 4 has a certain height and forms folds from the thin regions. Since these folds have a thinner thickness than other parts of the valve assembly 4, only the folds open when the valve assembly 4 allows gas flow, and the folds remain tightly closed when there is no gas flow. For ease of opening and closing of the thin region 42, the thin region 42 at least partially includes flexible material. After determining the form of the valve assembly 4, various limitations are placed on the valve assembly 4 to make it more effective in use. Among them, the thickness of at least part of the thin region 42 is set to be less than or equal to 5 mm (as shown in FIG. 6, where d≤5 mm). The thin region 42 is configured not to damage its material or performance during deformation, which means it maintains its functionality even after multiple openings and closings, thereby ensuring a longer service life. To ensure the one-way valve restricts airflow in one direction, the mask 1 is configured such that the second side 22 of the mask body 2 has an opening 221, and a projected area of the valve assembly 4 on the horizontal plane is greater than 30% of the area of the opening 221 (as shown in FIG. 7). The valve assembly 4 has two ends, and when there is no airflow, the projected areas of the two ends on a horizontal plane differ. This design allows the valve assembly 4 to be open when airflow is permitted in the direction of gas flow. One end of the valve assembly 4 is a slit, which means the valve assembly 4 has at least one slit provided in the middle of the thin region 42 and/or in the area where multiple thin regions 42 join. When the slit is set in the area where multiple thin regions 42 join, the valve assembly 4 typically has a larger airflow port 41.

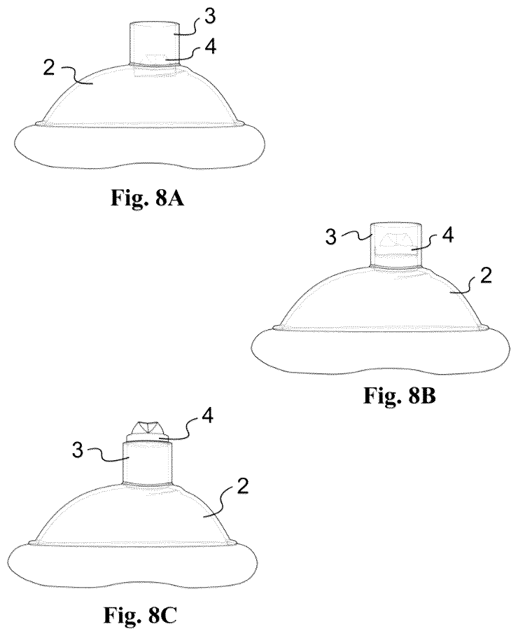

In this embodiment, the valve assembly 4 is provided within the cavity 33 of the connecting part 3. Therefore, the outer perimeter of the valve assembly 4 is less than or equal to the perimeter of the opening 221. The valve assembly 4 can be positioned anywhere within the cavity 33 (as shown in FIG. 8), and the central axis of the valve assembly 4 may coincide with or be parallel to the central axis of the connecting part 3. The valve assembly 4 can also be provided in a position within the through passage 331 but outside the cavity 33, which is common when the valve assembly 4 is not directly connected to the mask body 2 but through other additional components. To ensure the effectiveness of the valve assembly 4 during use, its position within the mask 1 is specified to ensure it is neither too close nor too far from the user's face. Thus, the distance from the valve assembly 4 to the first side 21 of the mask body 2 is set to be greater than one-tenth of the distance from the second side 22 to the first side 21 of the mask body 2. Additionally, the distance between the valve assembly 4 and the fourth side 32 of the connecting part 3 is set to be less than or equal to the distance between the first side 21 of the mask body 2 and the fourth side 32 of the connecting part 3. This more specifically defines the position of the valve assembly 4 within the mask 1 for effective and comfortable use.

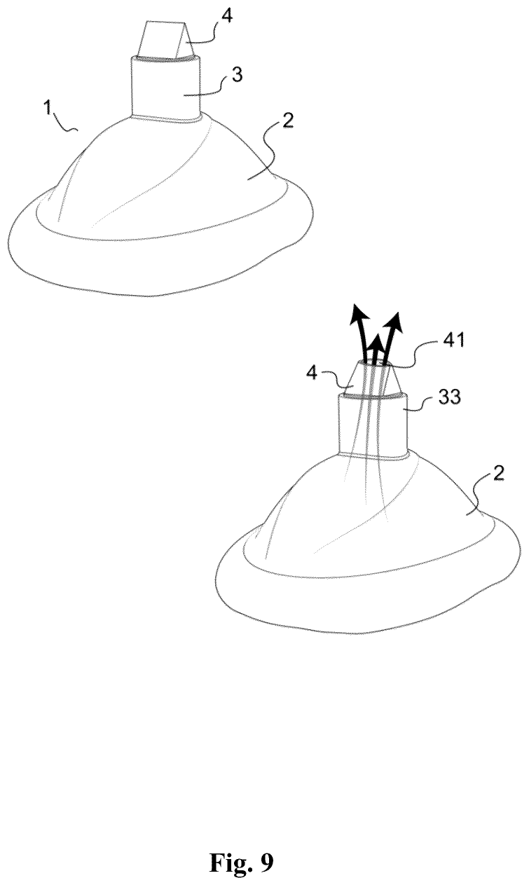

In some other embodiments, the flexible material of the valve assembly 4 forms a certain height but without folds (as shown in FIG. 9, which only illustrates one form in one embodiment).

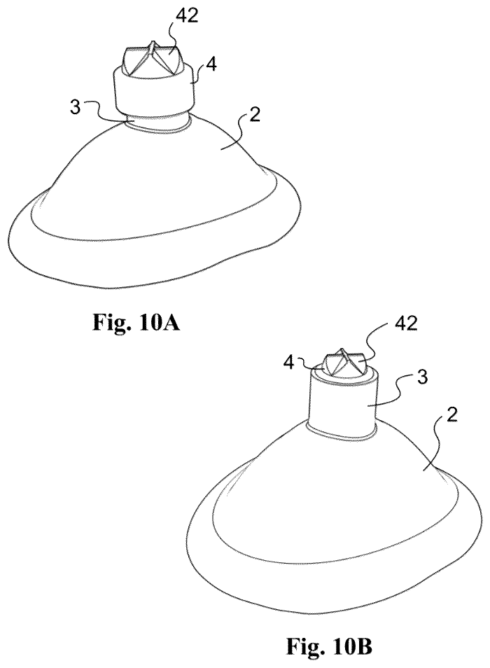

In other embodiments, the outer perimeter of the valve assembly 4 is greater than the perimeter of the opening 221 (as shown in FIG. 10A), meaning the valve assembly 4 surrounds the outer wall of the connecting part 3.

### EMBODIMENT 2

In this embodiment, the mask 1 includes: a mask body 2, a connecting part 3 and a valve assembly 4. The mask body 2 has a first side 21 that is configured to contact the user's face and a second side 22 opposite to the first side 21. The mask body 2 is configured to at least partially cover the user's entrance of a nasal and/or oral airway.

The connecting part 3 has a third side 31 to connect to the second side 22 and a fourth side 32 opposite to the third side 31. The connecting part 3 is configured to expel airflow from the mask body 2 or to deliver airflow into the mask body 2. The valve assembly 4, provided on the through passage 331 formed by extending on both sides of the connecting part 3, is configured to allow airflow to pass through the connecting part 3 in one direction only.

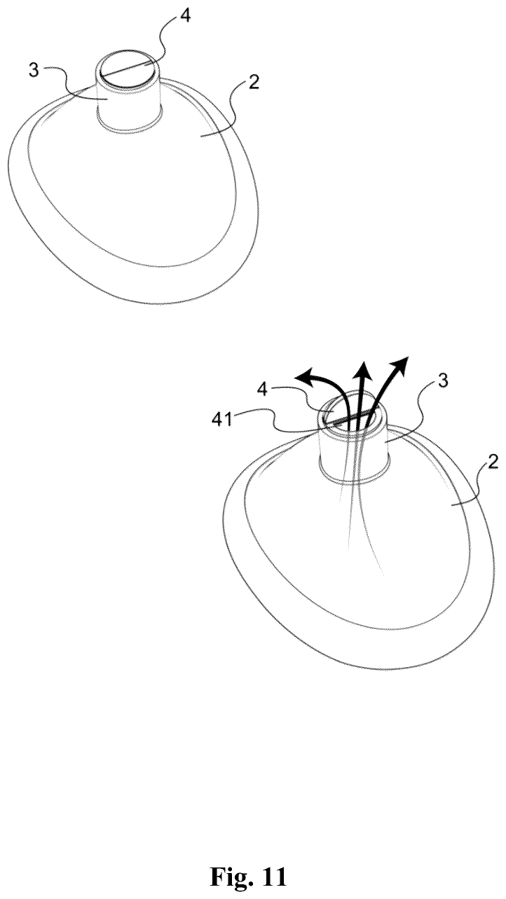

The difference between this embodiment and Embodiment 1 is that in this embodiment, the flexible part of the valve assembly 4 does not form a height and is in the form of a thin sheet. In this embodiment, the valve assembly 4 is divided into at least two parts, having a different structure from Embodiment 1 but performing the same function (as shown in FIG. 11). Specifically, the valve assembly 4 in this embodiment includes a baffle and a lifting part. The baffle and lifting part include at least two different materials. Typically, the baffle part is made of plastic, while the lifting part, which is the flexible part, is made of a softer material. The lifting part can also be interpreted as the thin region 42, having a thinner thickness than the baffle of the valve assembly 4 and being configured so that it does not damage its material or performance during deformation. As in Embodiment 1, the flexible part is also configured to form at least one airflow port 41 when the valve assembly 4 allows gas flow. However, in this case, the formation of the airflow port 41 does not rely on the flexible part but is formed by the baffle. Specifically, at least part of the lifting part is fixed to the baffle, and the unfixed part is the movable part that can be lifted. When the valve assembly 4 allows gas flow, the lifting part is blown open by the airflow, exposing the opening in the baffle, and the airflow exits through the opening of the baffle. When the valve assembly 4 does not allow gas flow, the lifting part is blocked by the baffle, thus forming a one-way valve that permits gas flow in only one direction. In such a case, at least one outer edge of the baffle is larger than the outer edge of the lifting part to block airflow in the opposite direction.

In some other embodiments, the baffle and the lifting part include the same material but with different thicknesses.

Furthermore, the technical features of the above embodiments can be combined as needed to obtain the mask 1 that includes all or some of the above technical features.

The implementation of a mask with a valve provided by this disclosure at least includes the following benefits:
1) The mask of this disclosure is versatile and applicable to multiple fields, including medical emergencies and home care. In the existing market, devices such as anti-choking devices, sputum suction devices, and emergency respirators all involve configurations where valves are used in conjunction with masks. However, there is room for improvement for these existing devices. For example, the existing anti-choking devices typically consist of a negative pressure generator, a one-way valve, and a mask. However, the one-way valve is not set up to connect directly with the mask, resulting in only a single channel at the junction between the negative pressure generator and the mask without any one-way valve to isolate the two. When the anti-choking device is activated, it first needs to expel excess air from the negative pressure generator to create a vacuum. At this time, because the channel between the negative pressure generator and the mask is not isolated, the air expelled by the negative pressure generator can easily enter the mask. This process contradicts the original purpose of the anti-choking device as it forces air into the mask that should be evacuating internal air. This forced air entry can push obstructions deeper into the user's airway and, in extreme cases, cause severe injuries like throat lacerations, presenting a hazard to the user. Therefore, by incorporating a one-way valve at an appropriate location on the mask and refining the design of the one-way valve to enhance its effectiveness and reliability, the mask provided by this disclosure can be used in existing devices to ensure the safety of devices like anti-choking systems. It guarantees the correct airflow during the vacuum creation process and prevents operational errors during this phase. Given the extensive use of one-way valves in conjunction with masks across multiple fields, the one-way valve-equipped mask of this disclosure improves various existing products, increasing the overall reliability of devices in multiple fields and enhancing the product experience for users.
2) In some devices within the aforementioned fields, there exists a one-way valve between the negative pressure generator and the mask. In such cases, using the mask with a one-way valve from this disclosure provides a secondary level of protection for the device. Specifically, the mask of this disclosure not only prevents the backflow of gases during the vacuum formation process but also maintains the stability of the vacuum more efficiently. In the event of an accidental device malfunction, the mask with the valve offers additional safety, making the device more secure and reliable. This dual mechanism is particularly crucial in high-demand or highly specialized fields, providing users with a safer and more effective respiratory support solution.
3) This disclosure also offers advantages when the valve and mask are integrated as a single module. a. For users, simply replacing the mask can enhance the overall device's safety, making it a very cost-effective choice. This is especially true for devices without isolation between the negative pressure generator and the mask, as replacing the original mask with a mask with a valve brings safety for users. b. Furthermore, when the valve and mask form a combinable module with a detachable connection, it allows users to choose between masks with or without a valve, thereby broadening their options and extending the adaptability of the mask of this disclosure to different application scenarios and needs. c. Moreover, compared to the combination of the valve with the negative pressure generator, the combination of the valve with the mask facilitates easier cleaning for the user. Due to the valve typically having more hygiene dead spots compared to other parts of the device, when cleaning is required, the mask itself, with its simple structure and bidirectional openings, makes the valve connected to the mask easier to clean thoroughly. This ensures that the device can maintain a good sanitary condition before and after each use, thereby enhancing the convenience of use.

The technical features of the above embodiments can be freely combined. For brevity, not all possible combinations of these features are described here. However, as long as the combinations do not introduce contradictions, they should be considered within the scope outlined by this disclosure.

The embodiments described above represent only a few of the possible implementations of the disclosure. Although the descriptions are specific and detailed, they should not be understood as limiting the scope of this disclosure. It should be noted that for one skilled in the art, various modifications and improvements can be made without departing from the concept of the disclosure, and these are also considered within the scope of protection of this disclosure. Therefore, the scope of protection for this disclosure should be determined by the appended claims.

It must be noted that as used herein and in the appended claims, the singular forms “a”, “an”, and “the” include their plural equivalents, unless the context clearly dictates otherwise.

1. A mask with a valve, configured to provide protection for a device with the mask in multiple fields, the mask comprising:
a mask body, having a first end configured to be in contact with a face of a user and a second end opposite the first end, wherein the first end of the mask body is configured to at least partially cover an entrance of a nasal and/or oral airway of the user;
a connecting part, having a third end adjacent to the second end and a fourth end opposite the third end, wherein the connecting part forms a through passage that is at least formed by sides of the connecting part that extend between the third end and the fourth end and that is configured to expel airflow from the mask body; and
a valve assembly, wherein the valve assembly is provided at least partially on the through passage, and wherein the valve assembly comprises the valve and is a one-way valve that is configured to allow the airflow to pass through the connecting part only in a direction of the airflow and prevents the airflow in an opposite direction through the connecting part, wherein the valve assembly includes a plurality of slits to allow the valve assembly to open when airflow is permitted in the direction of the airflow

2. The mask according to claim 1, wherein at least one cross-section of the connecting part has a shape of a circle, an ellipse, or a polygon.

3. The mask according to claim 2, wherein at least part of the connecting part has an unbent shape.

4. The mask according to claim 1, wherein the valve assembly has two ends, and when there is no airflow, an area of one end differs from an area of another end.

5. The mask according to claim 1, wherein the valve assembly includes one or more of the following materials: silicone, plastic, or metal.

6. The mask according to claim 1, wherein the first end of the mask body further comprises a flexible pad, and the flexible pad is configured to form a seal with the face of the user.

7. A mask with a valve, configured to provide protection for a device with the mask in multiple fields, the mask comprising:
a mask body, having a first end configured to be in contact with a face of a user and a second end opposite the first end, wherein the mask body is configured to at least partially cover an entrance of a nasal and/or oral airway of the user;
a connecting part, having a third end adjacent to the second end and a fourth end opposite the third end, wherein the connecting part forms a through passage that is at least formed by sides of the connecting part that extend between the third end and the fourth end and that is configured to expel airflow from the mask body;
a valve assembly, wherein the valve assembly is provided at least partially on the through passage, wherein the valve assembly comprises the valve and is a one-way valve that is configured to allow the airflow to pass through the connecting part in one direction only and prevents the airflow in an opposite direction through the connecting part, wherein the one-way valve is provided on top of the fourth end of the connecting part, and
wherein an opening is provided on the second end of the mask body, and a projected area of the valve assembly on a horizontal plane is greater than 30% of an area of the opening.

8. (canceled)

9. (canceled)

10. The mask according to claim 7, wherein the valve assembly is configured to allow the airflow to flow from the first end towards the fourth end only.

11. The mask according to claim 7, wherein at least part of the valve assembly is connectable and secured to the connecting part.

12. The mask according to claim 11, wherein a connection of at least part of the valve assembly to the connecting part is achieved through one or more of the following: adhesive bonding, welding, or mechanical structural connection.

13. A mask with a valve, configured to provide protection for a device with the mask in multiple fields, the mask comprising:
a mask body, having a first end configured to be in contact with a face of a user and a second end opposite the first end, wherein the mask body is configured to at least partially cover an entrance of a nasal and/or oral airway of the user;
a connecting part, having a third end adjacent to the second end and a fourth end opposite the third end, wherein the connecting part forms a through passage that is at least formed by sides of the connecting part that extend between the third end and the fourth end and that is configured to expel airflow from the mask body;
a valve assembly, wherein the valve assembly is provided at least partially [in] on the through passage, wherein the valve assembly comprises the valve and is a one-way valve that is configured to allow the airflow to pass through the connecting part in one direction only and prevents the airflow in an opposite direction through the connecting part, wherein the valve assembly includes a plurality of slits to allow the valve assembly to open when airflow is permitted in the direction of the airflow; and
wherein, during a process of unidirectional airflow, the valve assembly forms at least one airflow port, and a maximum area of the at least one airflow port is less than or equal to 38.485 cm².

14. The mask according to claim 13, wherein a central axis of the valve assembly coincides with or is parallel to a central axis of the connecting part.

15. The mask according to claim 13, wherein a distance from the valve assembly to the first end of the mask body is greater than one-tenth of a distance from the second end of the mask body to the first end.

16. The mask according to claim 13, wherein a distance between the valve assembly and the fourth end of the connecting part is less than or equal to a distance between the first end of the mask body and the fourth end of the connecting part.

17. (canceled)

18. A mask with a valve, configured to provide protection for a device with the mask in multiple fields, the mask comprising:
a mask body, having a first end configured to be in contact with a face of a user and a second end opposite the first end, wherein the mask body is configured to at least partially cover an entrance of a nasal and/or oral airway of the user;
a connecting part, having a third end adjacent to the second end and a fourth end opposite the third end, wherein the connecting part forms a through passage that is at least formed by sides of the connecting part that extend between the third end and the fourth end and that is configured to expel airflow from the mask body;
a valve assembly, wherein the valve assembly is provided at least partially on the through passage, wherein the valve assembly comprises the valve and is a one-way valve that is configured to allow the airflow to pass through the connecting part in one direction only and prevents the airflow in an opposite direction through the connecting part, wherein the one-way valve is provided on top of the fourth end of the connecting part;
wherein the valve assembly comprises at least one thin region,
wherein the at least one thin region has a thickness thinner than other parts of the valve assembly, and is configured to deform to allow unidirectional airflow through the valve assembly; and
wherein a thickness of at least part of the at least one thin region is less than or equal to 5 mm.

19. The mask according to claim 18, wherein the valve assembly includes at least one slit, and the at least one slit is plurality of slits are provided on a middle of the at least one thin region and/or at a joining area of multiple thin regions.

20. The mask according to claim 18, wherein the at least one thin region is configured so that it does not damage its material or performance during deformation.

21. The mask according to claim 18, wherein at least part of the at least one thin region includes a flexible material.

22. The mask according to claim 18, wherein the valve assembly and the connecting part are not integrally formed.

23. The mask according to claim 18, wherein the valve assembly and the connecting part are integrally formed.
